(19) United States
(12) Patent Application Publication
(10) Pub. No.: US 2021/0134409 A1
(43) Pub. Date: May 6, 2021

(54) REPORT MANAGEMENT SYSTEM

(71) Applicant: Konica Minolta, Inc., Tokyo (JP)

(72) Inventor: Kenichirou Suzuki, Tokyo (JP)

(73) Assignee: Konica Minolta, Inc., Tokyo (JP)

(21) Appl. No.: 17/083,649

(22) Filed: Oct. 29, 2020

(30) Foreign Application Priority Data
Oct. 30, 2019 (JP) 2019-196873

(51) Int. Cl. G16H 10/60; G16H 30/20; G16H 50/20

(52) U.S. Cl. CPC G16H 10/60; G16H 50/20; G16H 30/20

(57) ABSTRACT

A report management system includes: a master server that manages a report created in a medical facility according to multiple states defined in a report creation and approval flow; and a slave server that manages a duplicate of data managed in the master server and accessible from outside the medical facility. The slave server includes: a memory that stores a temporary report created based on an access from outside the medical facility; and a first hardware processor that generates a request for processing. The master server includes a second hardware processor that acquires the temporary report and the request for processing from the slave server, registers the temporary report as the report to be managed based on the request for processing, and changes a state of the report to be managed to a state that is selected from among the multiple states based on the request for processing.

### CROSS-REFERENCE TO RELAYED APPLICATIONS

The entire disclosure of Japanese Patent Application No. 2019-196873 filed on Oct. 30, 2019 is incorporated herein by reference.

### BACKGROUND

### Technical Field

The present invention relates to a report management system.

### Description of the Related Art

In the medical field, patients undergo imaging examinations of various modalities. For a medical image(s) taken by a modality, an attending doctor requests a diagnostician (diagnostic radiologist, doctor who interprets medical images) to give image diagnosis, and the diagnostician creates an image interpretation report. The attending doctor makes diagnosis referring to the image interpretation report created by the diagnostician.

The status (interpretation status) of the image interpretation report transitions between “uninterpreted,” “approval pending (interpreted),” “approved,” etc. according to a predetermined report creation and approval flow. The report system manages the interpretation reports with the report statuses.

There has been a technique in which the status of an image interpretation report is set to “pending” or “approved (confirmed)” according to the user setting when a report of image interpretation results that has been outsourced to an external faculty is received by a server PC (see JP 2016-197313 A), for example.

In addition to a PACS (Picture Archiving and Communication System) and an on-premise server including the report system (hereinafter referred to as a master server), a backup server (hereinafter referred to as a slave server) that backs up image and report data stored in the master server as needed outside the medical facility is prepared for a hardware error, a disaster, etc. Being outside the medical facility, the slave server is used not only temporarily in case of failure of the master server, but also as a server connected from outside the medical facility when the attending doctor or the diagnostician is absent.

Using such a system, in emergency occasions, the attending doctor accesses to the slave server on the Internet from an external terminal to refer to the images and past reports, and explains about treatment by phone, chat, etc. to an emergency doctor on site. The attending doctor creates a report afterwards inside the medical facility, but in that case, he/she needs to recollect the interpretation outside the medical facility to write a report, and select and edit the image(s) to attach to the report. Therefore, a formal report is desirably created at the timing of the interpretation outside the medical facility.

However, there are two issues in creating a report in image interpretation outside the medical facility.

First, a relationship between the server in the medical facility and the server outside the medical facility is master and slave, and thus the images and reports are always updated in the master server first and then in the slaver server. The consistency of data is maintained between the servers, but when the slave server outside the medical facility is directly used to create a report, the consistency of data may not be maintained in some cases. Thus, it is necessary that reports created outside the medical facility are not uploaded in the slave server in which reference images are stored, but are registered in the master server before reflected in the slave server.

Second, there may be a trouble in transition of the status in the report creation and approval flow when the report data created in an environment outside the medical facility is taken into the master server. The reports created outside the medical facility should not always be in the “completed (approval pending)” or “approved” status. In the technique of JP 2016-197313 A, when the report created in an external faculty for image interpretation is taken in, the status may transition to “approval pending” or “approved.” But actually, the status transition is determined more precisely. Specifically, the transition target that is set when the report data is taken in to the master server changes in the report creation and approval flow depending on various conditions such as practical rules in the medical facility, functions of the master server used in the medical facility, and statuses of those who interprets images outside the medical facility.

For example, in the case where a specific image(s) need to be rechecked with the environment for image interpretation (high definition/brightness) in the medical facility, the report is not to be in the “approved” status when the report data created outside the medical facility is taken in to the master server. This is because it is necessary to check whether image interpretation is correctly performed, as the environment for image interpretation is different from inside the medical facility when the report is created in an external environment, on a small-size tablet, etc.

The same can be applied in the case a viewer software provided by the master server is to be used in report creation as a report template is fixed for a specific report, in the case where an diagnostician present in the medical facility is to be requested to make image interpretation for check after image interpretation outside the medical facility, or in the case where a report created by an external faculty is to be in the status of “completed (approval pending),” and a report created in a hurry by an attending doctor is to be in the status of “in process (to resume report creation back in the medical facility in the continuation)”.

### SUMMARY

One or more embodiments of the present invention enable taking in the report created outside the medical facility to the server inside the medical facility while maintaining the consistency of data between the server in the medical facility and the server outside the medical facility.

A report management system of one or more embodiments includes:

a master server that manages a report created in a medical facility according to multiple states defined in a report creation and approval flow; and

a slave server that manages a duplicate of data that is managed in the master server and that is accessible from outside the medical facility,

wherein the slave server includes:
a memory that stores a temporary report that is created based on an access from outside the medical facility; and
a first hardware processor that generates a request for processing that is performed when the master server takes in the temporary report,

wherein the master server includes a second hardware processor that:
acquires the temporary report and the request for processing concerning the temporary report from the slave server; and
registers the acquired temporary report as a report to be managed in the master server based on the acquired request for processing and changes a state of the report to be registered into a state that is selected from among the multiple states according to the request for processing.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 shows a system configuration of a report management system according to one or more embodiments of the present invention;

FIG. 2 shows a configuration of a temporary report configuration file according to one or more embodiments;

FIG. 3 is a block diagram showing a configuration of a client terminal according to one or more embodiments;

FIG. 4 shows structures of a mater database of a master server, and a slave database of a slave server according to one or more embodiments;

FIG. 5 shows a data structure of a report status definition table according to one or more embodiments;

FIG. 6 is a report status transition diagram according to one or more embodiments;

FIG. 7 shows a data structure of a report trigger definition table according to one or more embodiments;

FIG. 8 shows a data structure of a report status transition definition table according to one or more embodiments;

FIG. 9 shows a data structure of a user definition table according to one or more embodiments;

FIG. 10 show a data structure of a role definition table according to one or more embodiments;

FIG. 11 shows a data structure of a role authority table according to one or more embodiments;

FIG. 12 shows a data structure of a report status transition slave control definition table according to one or more embodiments;

FIG. 13 shows a data structure of an examination table according to one or more embodiments;

FIG. 14 shows a data structure of a series table according to one or more embodiments;

FIG. 15 shows a data structure of an image table according to one or more embodiments;

FIG. 16 shows a data structure of a report table according to one or more embodiments;

FIG. 17 shows a data structure of a temporary report storage table according to one or more embodiments;

FIG. 18 shows a data structure of a check request task queue table according to one or more embodiments;

FIG. 19 shows a data structure of a temporary report storage database of the slave server according to one or more embodiments;

FIG. 20 shows a data structure of a check request response queue table according to one or more embodiments;

FIG. 21 is a ladder chart showing an image check request process according to one or more embodiments;

FIG. 22 shows an example of an examination list screen displayed on the client terminal inside a medical facility according to one or more embodiments;

FIG. 23 shows an example of a request target selection screen displayed on the client terminal inside the medical facility according to one or more embodiments;

FIG. 24 is a ladder chart showing a temporary report creation process according to one or more embodiments;

FIG. 25 shows an example of a check request notification screen displayed on the client terminal outside the medical facility according to one or more embodiments;

FIG. 26 shows an example of a viewer screen displayed on the client terminal outside the medical facility according to one or more embodiments;

FIG. 27 shows an example of a report screen displayed on the client terminal outside the medical facility according to one or more embodiments;

FIG. 28 is a ladder chart showing a temporary report acquisition request process according to one or more embodiments;

FIG. 29 is a ladder chart showing a temporary report check process according to one or more embodiments;

FIG. 30 shows an example of a note notification screen displayed on the client terminal inside the medical facility according to one or more embodiments; and

FIG. 31 shows an example of a report screen displayed on the client terminal inside the medical facility according to one or more embodiments.

### DETAILED DESCRIPTION OF THE EMBODIMENTS

Hereinafter, embodiments of a report management system according to the present invention are described with reference to the drawings. However, the scope of the invention is not limited to the disclosed embodiments.

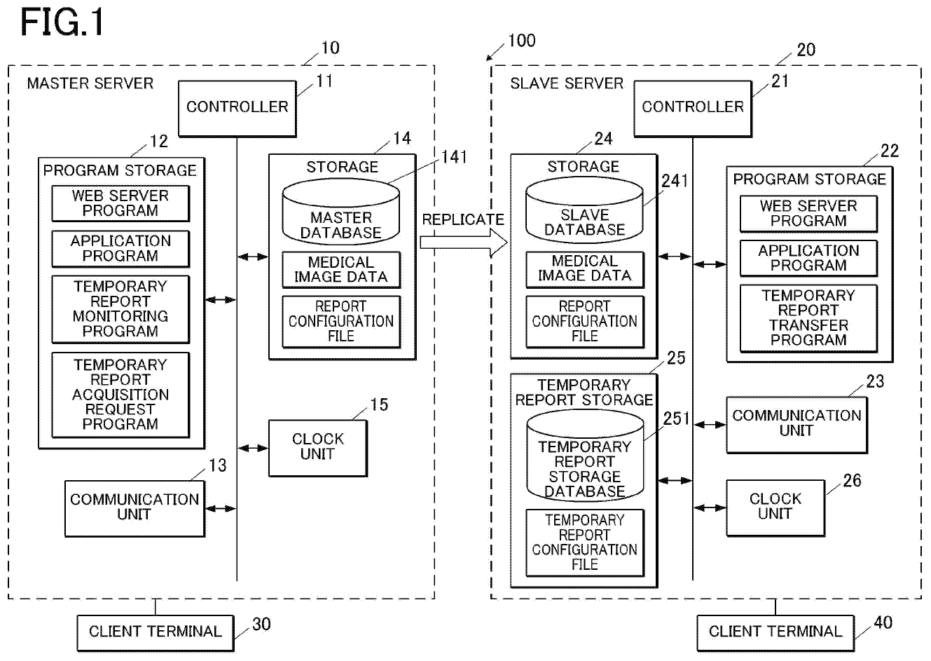

FIG. 1 shows a system configuration of a report management system 100. As shown in FIG. 1, the report management system 100 includes a master server (on-premise server) 10 installed inside a medical facility, and a slave server (back-up server) 20 installed outside the medical facility. The master server 10 and the slave server 20 are connected to one another via a communication network such as the Internet for data communication.

The master server 10, which is accessible in the medical facility, manages image data (medical image data) of medical images generated by modalities, and report data for each examination. The master server 10 includes a PACS (Picture Archiving and Communication System) and a conventional report system. The master server 10 manages reports generated in the medical facility according to statuses defined in a report creation and approval flow. The report creation and approval flow is information on a flow (order) of steps defined with the status transition of the report, in which a report is generated for a medical image(s) and then approved.

The master server 10 includes a controller 11 (second hardware processor), a program storage 12, a communication 13, a storage 14, and a clock unit 15.

The controller 11, which includes a central processing unit (CPU) and a random access memory (RAM), centrally controls processing operations of the components of the master server 10. Specifically, the CPU retrieves various processing programs stored in the program storage 12, deploys them in the RAM, and executes various kinds of processing in cooperation with the programs.

The program storage 12 stores various processing programs executed by the controller 11. The program storage 12 stores, for example, a web server program, an application program, a temporary report monitoring program, and a temporary report acquisition request program.

The web server program is for executing functions as a web server that provides various web screens on a web browser by communication with the web browser installed in the client terminal 30 in the medical facility using an HTTP protocol.

The application program, which operates on the web server, is for providing an image viewer screen, a report screen, etc. to the client terminal 30 via the web browser for a user. The controller 11 refers to a master database 141 in response to an access from the client terminal 30 in the medical facility, and generates data of a screen displayed on the client terminal 30 on the basis of a target examination, image(s), report data, in cooperation with the application program.

The temporary report monitoring program is for regularly referring to a temporary report storage database 251 in the slave server 20.

The temporary report acquisition request program is for sending a request for acquisition of a temporary report configuration file to the slave server 20 and storing the acquired temporary report configuration file in the storage 14.

The communication unit 13, which includes a network interface, sends and receives data to and from an external device(s) connected via the communication network. For example, the communication unit 13 sends the information managed in the master server 10 to the slave server 20. The communication unit 13 acquires data concerning temporary reports from the slave server 20.

The storage 14, which includes a hard disk drive (HDD) and a non-volatile memory, stores various kinds of data. The storage 14 stores the master database 141, the medical image data, the report configuration files, etc.

The medical image data is a DICOM image(s) in conformity with the DICOM (Digital Image and Communications in Medicine) standard. Attribute information (patient information, examination information, etc.) is attached to the DICOM image(s), as written in the header of the image file of the medical image(s).

The report configuration file is report data of an image interpretation report created by image interpretation of the medical image(s). The report configuration file includes a report text (text data of the report), report structure information (a layout of the report, storage location and information concerning the text and image(s)), and image(s) for the report (image data to be attached to the report).

The clock unit 15, which includes a real time clock (RTC), outputs the present date and time measured with the RTC to the controller 11.

The master server 10 is set to a “master mode (normal mode).” In the master mode, the data in the database (master database 141) may be updated.

The slave server 20, which is accessible from outside the medical facility, manages duplication of the information managed in the master server 10.

The slave server 20 includes a controller 21 (first hardware processor), a program storage 22, a communication unit 23, a storage 24, a temporary report storage 25 (memory), and a clock unit 26.

The controller 21, which includes a CPU and a RAM, centrally controls processing operations of the components of the slave server 20. Specifically, the CPU retrieves various processing programs stored in the program storage 22, deploys them in the RAM, and executes various kinds of processing in cooperation with the programs.

The program storage 22 stores various processing programs executed by the controller 21. The program storage 22 stores, for example, a web server program, an application program, and a temporary report transfer program.

The web server program and the application program are the same as the web server program and the application program in the master server 10, but are different therefrom in that the destination of processing is the client terminal 40 outside the medical facility. The controller 21 refers to a slave database 241 in response to an access from the client terminal 40 in the medical facility, and generates data of a screen displayed on the client terminal 40 on the basis of a target examination, image(s), report data, in cooperation with the application program.

The temporary report transfer program is for sending a temporary report configuration file to the master server 10 in response to a request from the master server 10.

The communication unit 23, which includes a network interface, sends and receives data to and from an external device(s) connected via the communication network. For example, the communication unit 23 receives the information managed in the master server 10 from the master server 10. The communication unit 23 sends data concerning a temporary report to the master serer 10. The temporary report includes a provisional report, a memo, etc. briefly generated outside the medical facility.

The storage 24, which includes an HDD and a non-volatile memory, stores various kinds of data. The storage 24 stores the slave database 241, the medical image data, the report configuration file, etc. The slave database 241, the medical image data, and the report configuration file are synchronized duplicates of the master database 141, the medical image data, and the report configuration file in the storage 14 of the master server 10 (replication).

The temporary report storage 25, which includes an HDD and a non-volatile memory, stores data concerning the temporary reports created via access from outside the medical facility. That is, the temporary report storage 25 functions as a storing means to store the temporary reports. The temporary report storage 25 stores the temporary report storage database 251, the temporary report configuration file, etc.

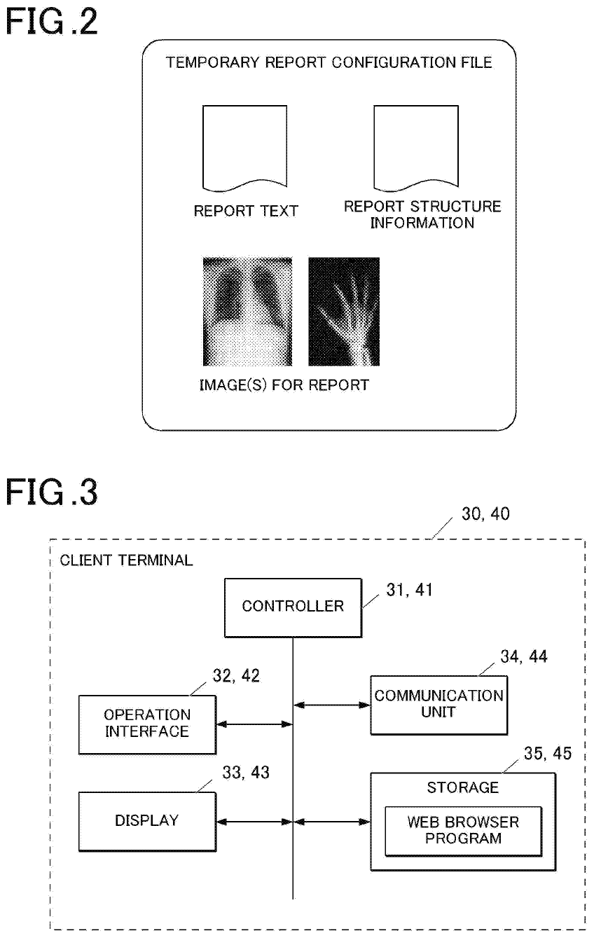

The temporary report configuration file is report data of a temporary report created by image interpretation of the medical image(s) outside the medical facility. As shown in FIG. 2, the temporary report configuration file includes a report text (text data of the temporary report), report structure information (a layout of the temporary report, storage location and information concerning the text and image(s)), and image(s) for the report (image data to be attached to the report).

The clock unit 26, which includes a real time clock (RTC), outputs the present date and time measured with the RTC to the controller 21.

The slave server 20 is set to a “slave mode.” In the slave mode, data update except in the temporary report storage database 251 is not permitted, and the slave database 241 only is allowed to be referred to.

The client terminal 30 is a computer device such as a personal computer (PC) used in the medical facility.

FIG. 3 shows a configuration of the client terminal 30. As shown in FIG. 2, the client terminal 30 includes a controller 31, an operation interface 32, a display 34, and a storage 35.

The controller 31, which includes a CPU and a RAM, centrally controls processing operations of the components of the client terminal 30. The CPU retrieves various processing programs stored in the program storage 35, deploys them in the RAM, and executes various kinds of processing in cooperation with the programs.

The operation interface 32, which includes a keyboard with cursor keys, letter and number input keys, various function keys, etc. and a pointing device such as a mouse, outputs key operations on the keyboard, operation signals input by the mouse operation, etc. to the controller 31.

The display 33, which includes a monitor of a liquid crystal display (LCD), shows various screens according to the display signals input by the controller 31.

The communication unit 34, which includes a network interface, sends and receives data to and from an external device(s) connected via the communication network.

The storage 35, which includes an HDD and a non-volatile memory, stores various kinds of data. For example, the storage 35 stores a web browser program for realizing a web browser.

The client terminal 40 is a computer device such as a PC used outside the medical facility.

As shown in FIG. 3, the client terminal 40 includes a controller 41, an operation interface 42, a display 43, a communication unit 44, and a storage 45. The configuration of the client terminal 40 is similar to that of the client terminal 30, and thus the description thereof is omitted.

Next, structures of the databases are described.

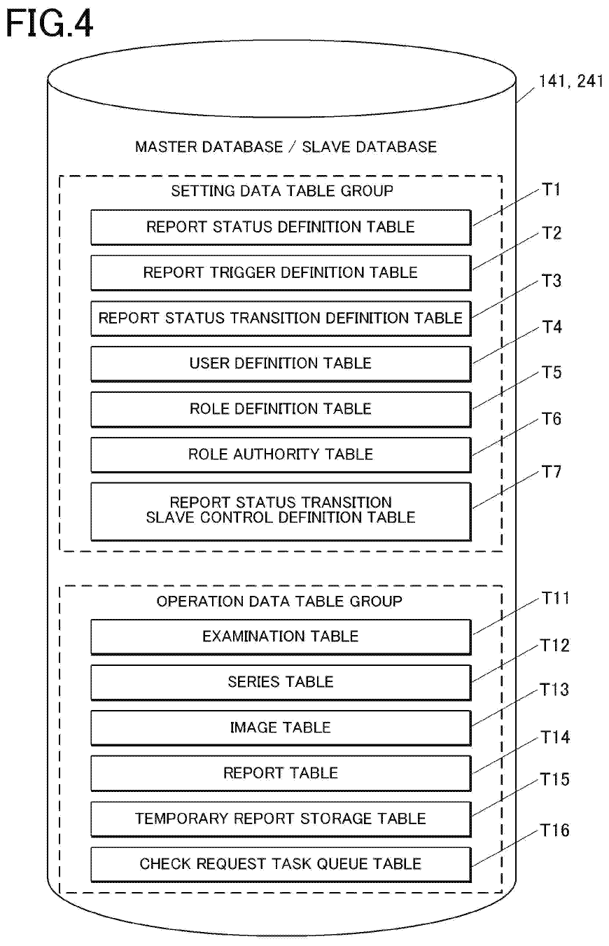

FIG. 4 shows a structure of the master database 141 in the storage 14 of the master server 10. The mater database 141 includes a report status definition table T1, a report trigger definition table T2, a report status transition definition table T3, a user definition table T4, a role definition table TS, a role authority table T6, a report status transition slave control definition table T7, an examination table T11, a series table T12, an image table T13, a report table T14, a temporary report storage table T15, and a check request task queue table T16. The tables T1 to T7 are a setting data table group prepared in advance (modification possible). The tables T11 to T16 are an operation data table group, which data is added and deleted to and from during operation of the system.

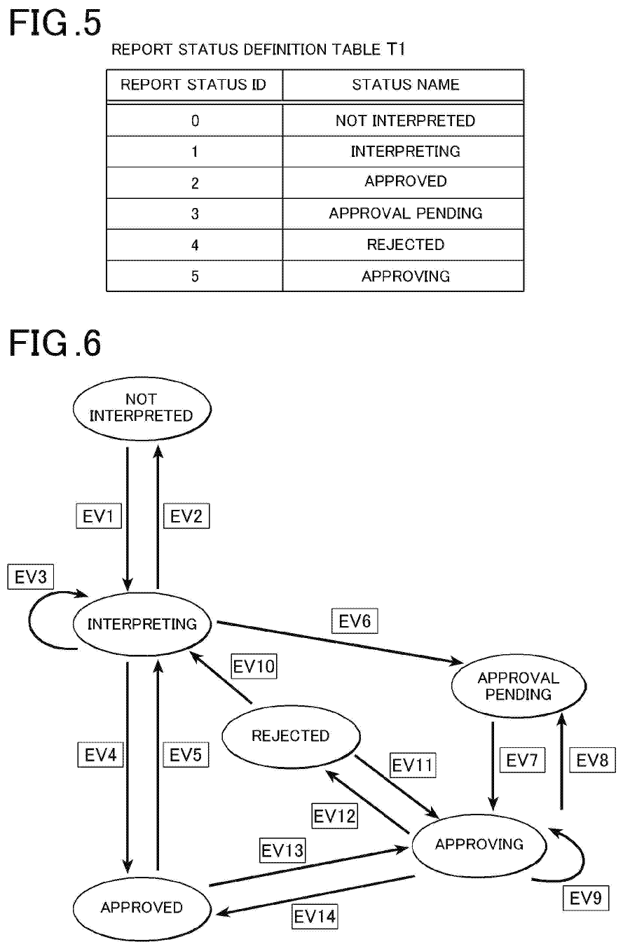

FIG. 5 shows a data structure of the report status definition table T1. The report status definition table T1 defines a status of report. In the report status definition table T1, a report status ID and a status name are associated with each other.

The report status is identification information given to the report status.

The status name is a name of the report status, and includes “uninterpreted (undone),” “interpreting (in progress),” “approved,” “approval pending,” “rejected,” and “approving.”

FIG. 6 is a report status transition diagram. The arrows between the statuses represent transition from one status to another. The arrows are associated respectively with triggers EV1 to EV14. The triggers EV1 to EV14 represent transition of the report statuses from the starting point to the ending point of arrow.

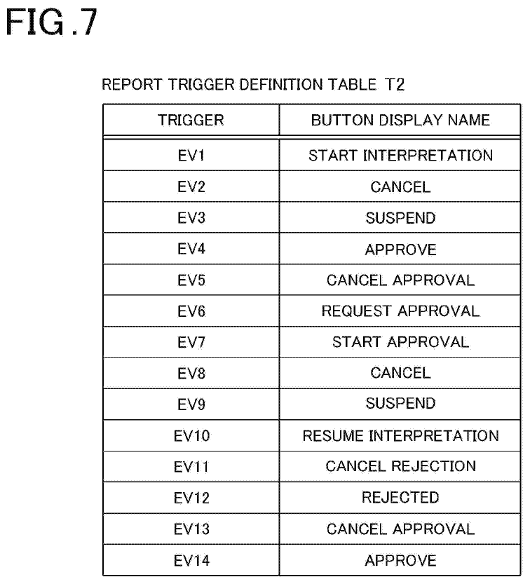

FIG. 7 shows a data structure of the report trigger definition table T2. The report trigger definition table T2 defines names of buttons displayed by the master server 10 for the triggers EV1 to EV14 that cause the report status transitions. In the report trigger definition table T2, the trigger and the display name of button are associated with each other.

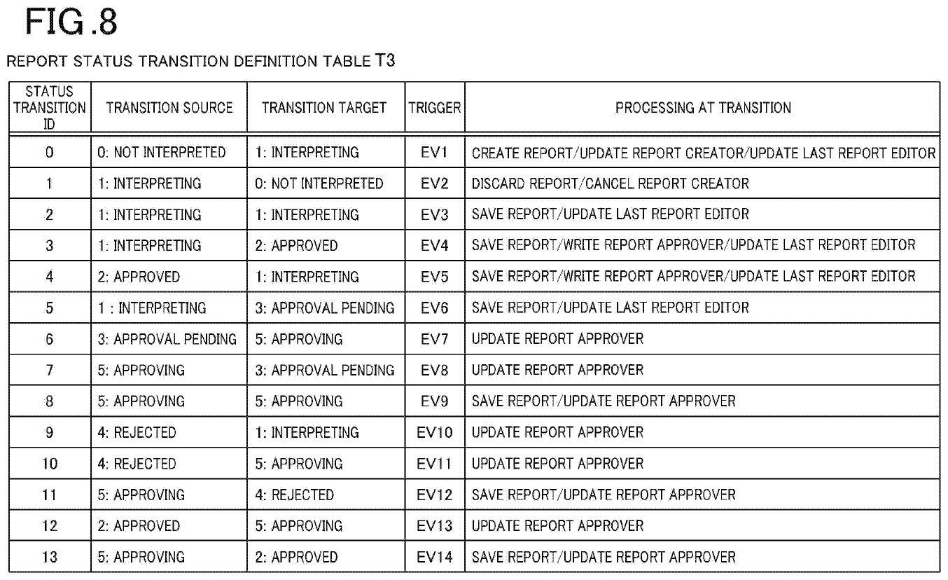

FIG. 8 shows a data structure of the report status transition definition table T3. The report status transition definition table T3 defines the transition target report statuses of the respective current report statuses (transition source) that are set when the buttons corresponding to the triggers are pressed. In the report status transition definition table T3, the status transition ID, the transition source, the transition target, the trigger, and the process at transition are associated with each other.

The status transition ID is identification information given to the report status transition.

The transition source is the report status ID corresponding to the current report status.

The transition target is the report status ID corresponding to the transition target report status.

The process at transition is a process executed at transition from the target source to the transition target.

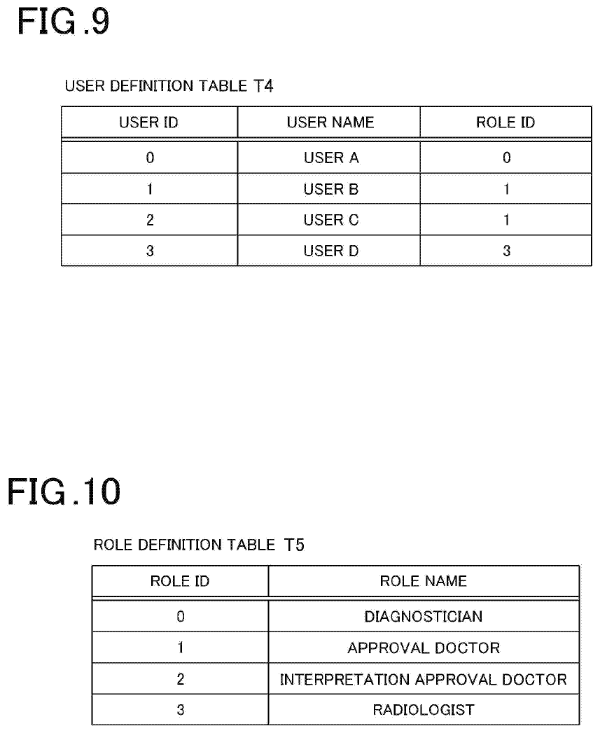

FIG. 9 shows a data structure of the user definition table T4. The user definition table T4 defines information concerning users of the report management system 100. In the user definition table T4, the user ID, the username, and the role ID are associated with each other.

The user ID is identification information given to a user.

The username is used when the user uses the report management system 100.

The role ID corresponds to a role (type/position) of the user in the system.

FIG. 10 shows a data structure of the role definition table T5. In the role definition table T5, the role ID and the role name are associated with each other.

The role name, the name of role in the system, is, for example, “diagnostician,” “approval doctor,” “image interpretation doctor,” or “radiologist.”

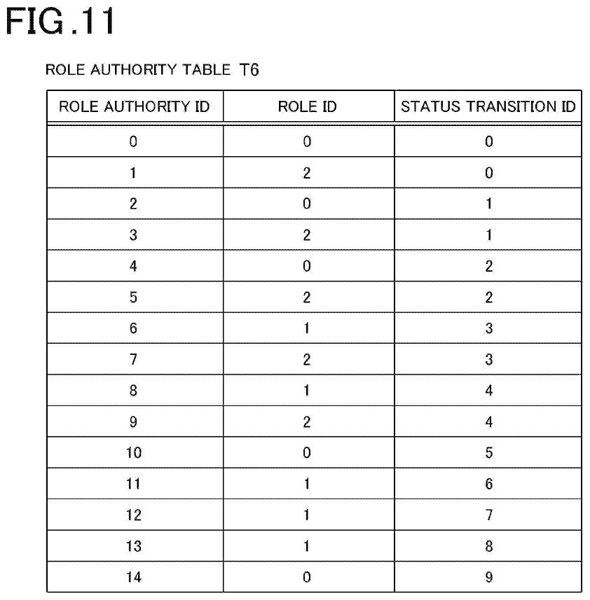

FIG. 11 shows a data structure of the role authority table T6. The role authority table T6 defines the status transition executable by each role (role authority). In the role authority table T6, the role authority ID, the role ID, and the status transition ID are associated with each other.

The role authority ID is identification information given to each role authority.

The status transition ID corresponds to the status transition executable by the role corresponding to the role ID.

For example, the role authority ID “0” indicates that the “diagnostician” whose role ID is “0” can execute the transition “from the uninterpreted to interpreting” whose status transition ID “0.”

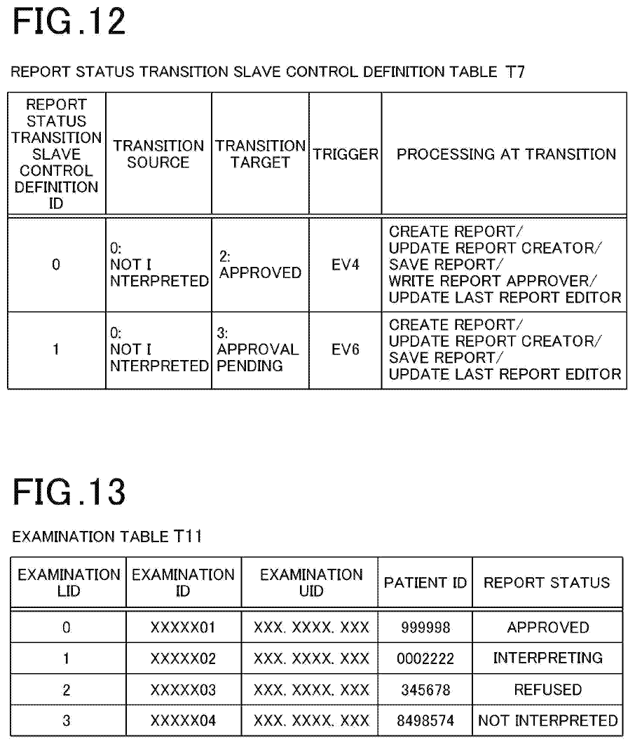

FIG. 12 shows a data structure of the report status transition slave control definition table T7. The report status transition slave control definition table T7 defines the transition target for a temporary report generated using the slave server 20. In the report status transition slave control definition table T7, the report status transition slave control definition ID, the transition source, the transition target, and the process at transition are associated with each other.

The report status transition slave control definition ID is identification information given to the control method (processing request) of the status transition set for the temporary report.

The transition source is the report status ID corresponding to the report status before the temporary report is created in the slave server 20.

The transition target is the report status ID corresponding to the transition target report status.

The trigger corresponds to the button displayed when the transition command for the transition to the target report status is received.

The process at transition is a process executed at transition from the target source to the transition target.

FIG. 13 shows a data structure of the examination table T11. In the examination table T11, the examination LID, the examination ID, the examination UID, the patient ID, and the report status are associated with each other.

The examination LID is management information for specifying an examination.

The examination ID is identification information given to the examination.

The examination UID is identification information given to the examination in conformity with the DICOM standard.

The patient ID is identification information given to the patient undergoing the examination.

The report status is a status of image interpretation of the report created for a medical image(s) taken in the examination.

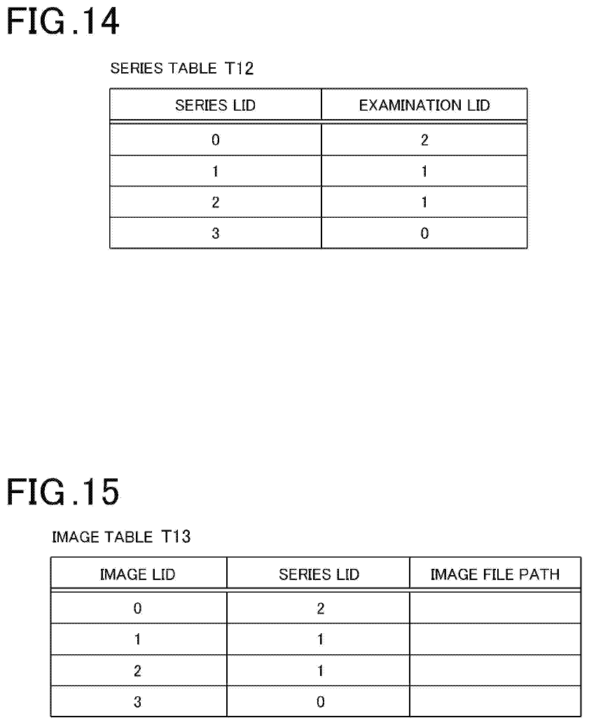

FIG. 14 shows a data structure of the series table T12. In the series table T12, the series LID and the examination LID are associated with each other.

The series LID is management information for specifying the series.

The examination LID is of the examination including the series.

FIG. 15 shows a data structure of the image table T13. In the image table T13, the image LID, the series LID, and the image file path are associated with each other.

The image LID is management information for specifying medical image data.

The series LID is of the series including the medical image data.

The image file path is a storage location of the file of the medical image data.

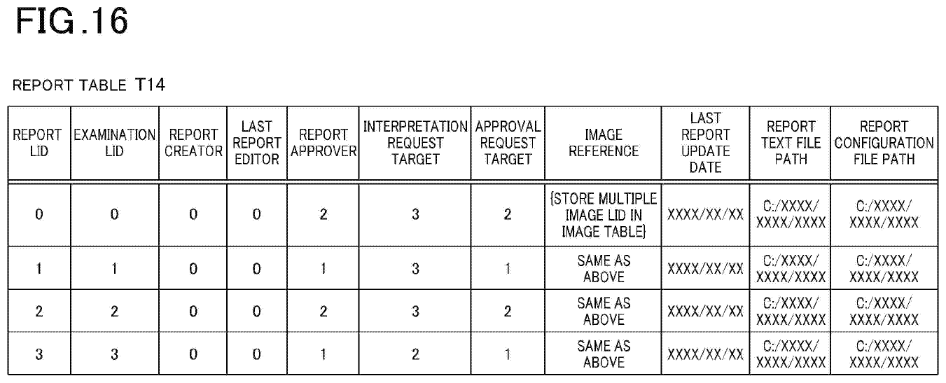

FIG. 16 shows a data structure of the report table T14. In the report table T14, the report LID, the examination LID, the report creator, the last report editor, the report approver, the image interpretation request target, the approval request target, the image reference target, the last report update date, the report text file path, and the report configuration file path are associated with each other.

The report LID is management information for specifying a report.

The examination LID is of the examination in which the target medical image(s) for the report is generated.

The report creator is the user ID of the user who created the report.

The last report editor is the user ID of the user who edited the last report.

The report approver is the user ID of the user who approved the report.

The image interpretation request target is the user ID of the user who requested image interpretation.

The approval request target is the user ID of the user who is requested to approve image interpretation.

The image reference target is the image LID corresponding to the medical image for the report. There may be multiple image reference targets.

The last report update date is the date when the last report is updated.

The report text file path is a storage location of the file of the report text included in the report configuration file.

The report configuration file path is a storage location of the report configuration file.

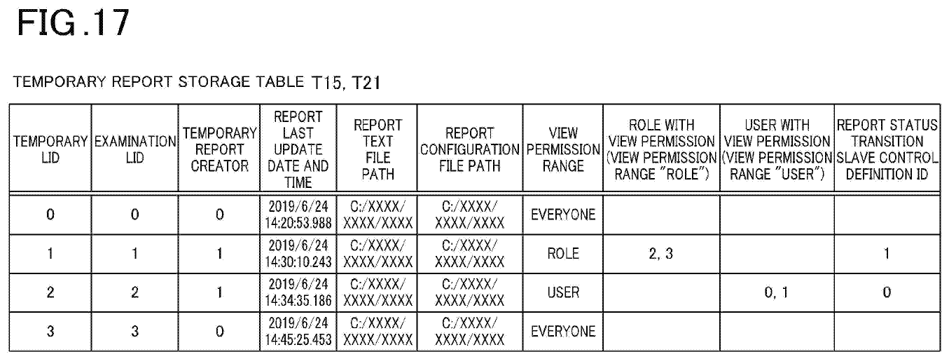

FIG. 17 shows a data structure of the temporary report storage table T15. In the temporary report storage table T5, the temporary report LID, the examination LID, the temporary report creator, the last report update date and time, the report text file path, the report configuration file path, the viewable range, the role with view permission, the user with view permission, and the report status transition slave control definition ID are associated with each other.

The temporary report LID is management information for specifying a temporary report.

The examination LID is of the examination in which a medical image(s) for the temporary report is generated.

The temporary report creator is the user ID of the user who created the report.

The last report update date and time is the date and time when the temporary report is updated for the last time.

The report text file path is a storage location of the file of the report text included in the temporary report configuration file.

The report configuration file path is a storage location of the temporary report configuration file.

The viewable range is a range of users who can view the temporary report. A range named “EVERYONE” is registered in the case where all the users can view the temporary report, a range named “ROLE” in the case where the viewable range is managed by role, and a range “USER” in the case where the viewable range is managed by user.

The role with view permission is the role ID which is granted the permission for view with the range “ROLE.”

The user with view permission is the user ID which is granted the permission for view with the range “USER.”

The report status transition slave control definition ID is identification information given to the control method (processing request) of the status transition set for the temporary report.

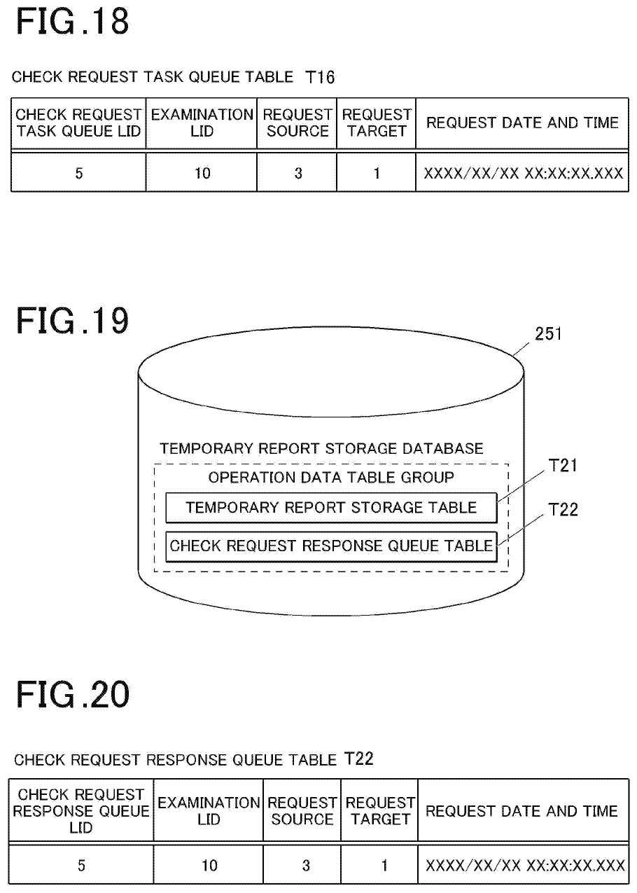

FIG. 18 shows a data structure of the check request task queue table T16. In the check request task queue table T16, the check request task queue LID, the examination LID, the request source, the request target, and the request date and time are associated with each other.

The check request task queue LID is management information for specifying a record (request for checking a medical image(s)) in the check request task queue table T16.

The examination LID is of the examination in which the target medical image(s) for the check request is generated.

The request source is the user ID of the user from whom check of the medical image(s) is requested.

The request target is the user ID of the user who is requested to check the medical image(s).

The request date and time is when the check of the medical image(s) is requested.

The data stored in the storage 24 of the slave server 20 is the replicate of the data in the master server 10. Thus, the configuration of the slave database 241 is similar to that of the master database 141 as shown in FIG. 4.

FIG. 19 shows a structure of the temporary report storage database 251 of the temporary report storage 25 of the slave server 20. The temporary report storage database 251 includes a temporary report storage table T21 and a check request response queue table T22. The temporary report storage table T21 and the check request queue table T22 are an operation data table group which data is added and deleted to and from during operation of the system.

The data configuration of the temporary report storage table T21 is similar to that of the temporary report storage table T15 as shown in FIG. 17.

FIG. 20 shows a data configuration of the check request response queue table T22. In the check request response queue table T22, the check request response queue LID, the examination LID, the request source, the request target, and the request date and time are associated with each other.

The check request response queue LID is management information for specifying a record (that received a response to a check request) in the check request response queue table T22.

The examination LID, the request source, the request target, and the request date and time are similar to those in the check request task queue table T16 shown in FIG. 18.

The controller 11 of the master server 10 sends the data of the master database 141, the medical image data, and the report configuration file data to the slave server 20 via the communication unit 13, regularly or when data is modified in the storage 14.

The controller 21 of the slave server 20 acquires the data in the storage 14 sent from the master server 10 via the communication unit 23, and stores the data in the storage 24 as the data of the slave database 241, the medical image data, and the report configuration file data. In this way, the storage 24 stores the replicate of the data stored in the storage 14 of the master server 10.

The controller 21 of the slave server 20 generates a request for processing of the master server 10 taking in the temporary report. That is, the controller 21 functions as a generating means. Specifically, the controller 21 generates a request for processing in response to an operation received from the client terminal 40 outside the medical facility. The “request for processing” corresponds to the “report status transition slave control definition ID” of a record added to the temporary report storage table T21 in the temporary report storage database 251 when the temporary report is generated in the slave server 20.

The controller 11 of the master server 10 acquires the temporary report and a request for processing of the concerning temporary report from the slave server 20. That is, the controller 11 functions as an acquiring means.

On the basis of the request for processing acquired from the slave server 20, the controller 11 registers the temporary report acquired from the slave server 20 as a report to be managed in the master server 10, and causes the status of the concerning registered report to be in a status according to the request for processing among multiple statuses. That is, the controller 11 functions as a taking-in means.

Specifically, the controller 11 acquires the “report status transition slave control definition ID” corresponding to the temporary report from the slave server 20, refers to the report status transition slave control definition table T7 in the master database 141, and acquires the “transition target” associated with the “report status transition slave control definition ID.” The controller 11 thereby specifies the transition target of the report status that is set when the master server 10 takes in the temporary report. That is, the request for processing includes information for specifying the transition target of the report status that is set when the maser server 10 takes in the temporary report (the report status transition slave control definition ID).

The controller 11 of the master server 10 refers to the examination table T11 of the master database 141 and acquires the “report status” associated with the “examination LID” of the concerning examination, when a viewer screen/report screen concerning the examination designated on the client terminal 30 is displayed on the display 33 of the client terminal 30. The controller 11 acquires the “role ID” of the user of the client terminal 30 from the user definition table T4 in the master database 141, refers to the role authority table T6 in the master database 141, and acquires the “status transition ID” associated with the acquired “role ID.” The controller 11 refers to the report status transition definition table T3 in the master database 141, and acquires a “trigger” associated with the current “report status” in the “transition target” column and with the “status transition ID” permitted to the user of the client terminal 30. The controller 11 refers to the report trigger definition table T2 in the master database 141, and displays the “button display name” associated with the acquired “trigger” on the screen of the client terminal 30.

Next, the operations executed in the report management system 100 are described.

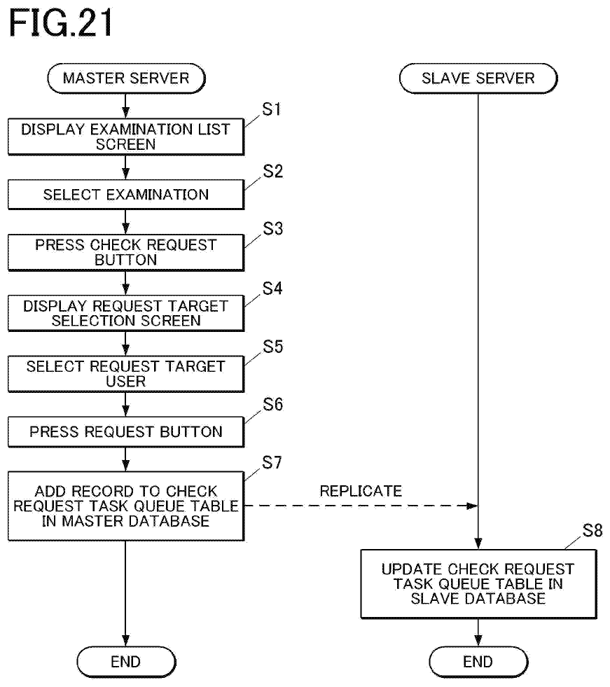

FIG. 21 is a ladder chart showing the image check request process. In the image check request process, check of a medical image(s) is requested from inside the medical facility to outside the medical facility.

First, when the user (doctor in the medical facility) inputs the URL for accessing the master server 10 on the web browser via the operation interface 32 in the client terminal 30 inside the medical facility, the controller 31 accesses the master server 10 based on the input URL via the communication unit 34.

In the master server 10, the controller 11 sends data for displaying the login screen via the communication unit 13 to the client terminal 30. The data for displaying the various web screens such as a login screen sent to the client terminal 30 by the web server function of the master server 10 includes HTML, style sheets, image data, and scripts for executing predetermined processes.

In the client terminal 30, the login screen is shown on the display 33. When the user inputs user ID on the login screen via the operation interface 32, the controller 31 sends the input user ID via the communication unit 34 to the master server 10.

In the master server 10, when the user ID is received via the communication unit 13, the controller 11 specifies the user (login user) accessing the master server 10.

The controller 11 of the master server 10 displays the examination list screen on the display 33 of the client terminal 30 (Step S1). Specifically, the controller 11 reads out the data of each examination (examination information, patient information, etc. associated with the examination LID of each examination) from the examination table T11, etc. of the master database 141, and generates data for displaying the examination list screen.

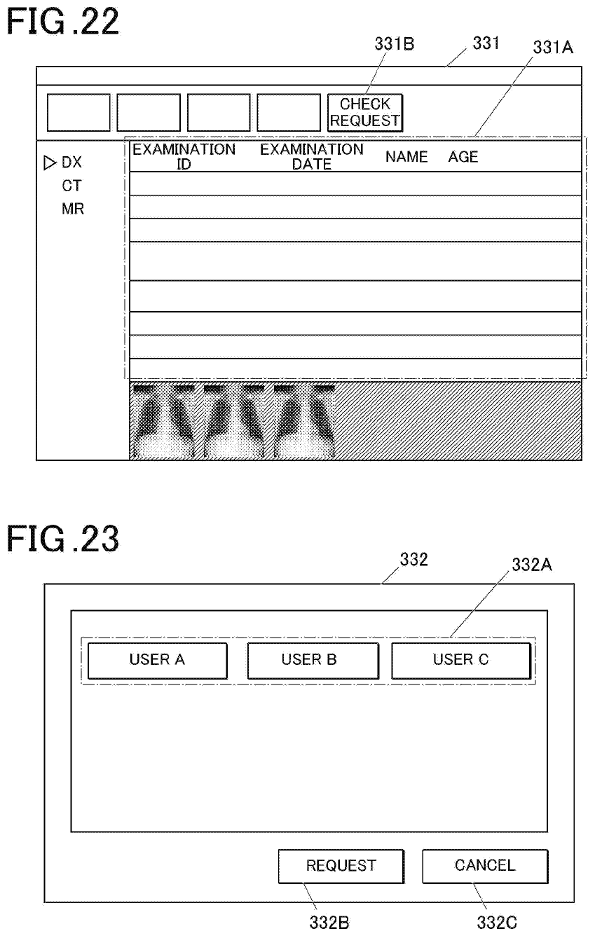

FIG. 22 shows an example of the examination list screen 331 to be displayed on the display 33 of the client terminal 30. The examination list screen 331 includes an examination list display area 331A and a “check request” button 331B.

When the user, on the client terminal 30, selects an examination in which an image(s) to be checked outside the medical facility is taken from the examinations shown in the examination list display area 331A via the operation interface 32 (Step S2) and presses the check request button 331B (Step S3), the controller 11 of the master server 10 shows the request target selection screen on the display 33 of the client terminal 30 (Step S4). Specifically, the controller 11 reads out the data for each user from the user definition table T4 of the master database 141 and generates data for displaying the request target selection screen.

FIG. 23 shows an example of the request target selection screen 332 displayed on the display 33 of the client terminal 30. The request target selection screen 332 includes a request target option display area 332A, a “request” button 332B, and a “cancel” button 332C.

When the user, on the client terminal 30, selects a user (request target user) to check the image among the users (doctors) shown in the request target option display area 332A via the operation interface 32 (Step S5) and presses the request button 332B (Step S6), the controller 11 of the master server 10 adds a record to the check request task queue table T16 in the master database 141 (Step S7). Specifically, the controller 11 assigns a new “check request task queue LID” in the check request task queue table T16 as a new record. The controller 11 enters the examination LID of the examination selected at Step S2 in the “examination LID” column, the user ID of the login user in the “request target” column, the user ID of the request target user selected at Step S5 in the “request target” column, and the present date and time acquired from the clock unit 15 in the “request date and time” column.

The data in the master database 141 of the master server 10 is constantly replicated in the slave database 241 of the slave server 20, and thus the check request task queue table T16 in the slave database 241 is also updated (Step S8).

The image check request process is now completed.

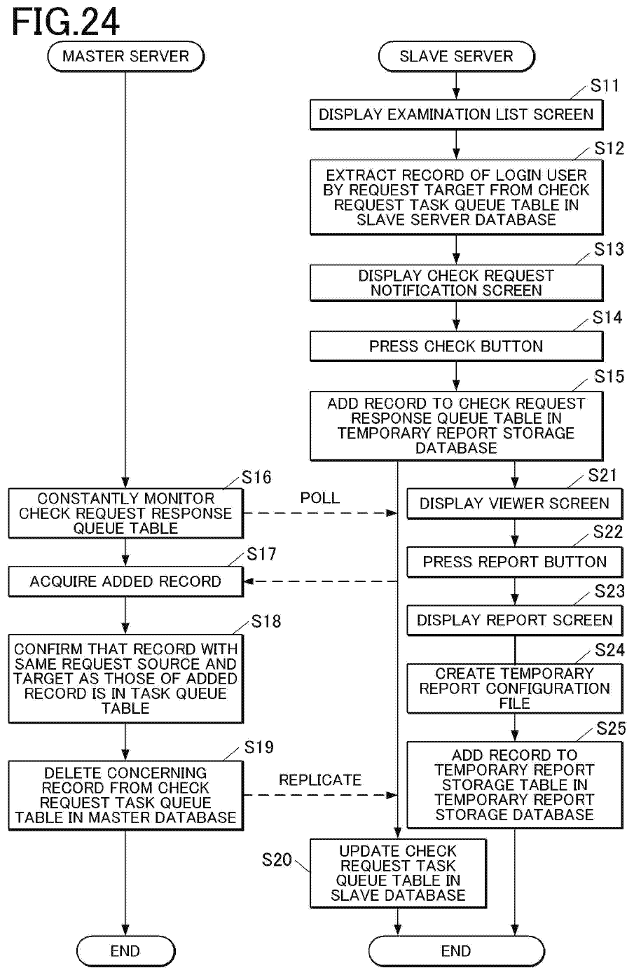

FIG. 24 is a ladder chart showing the temporary report creation process. In the temporary report creation process, a temporary report is created outside the medical facility.

As a premise of this process, the slave server 20 is accessed from the client terminal 40 outside the medical facility for the login process. The controller 21 of the slave server 20 specifies the user (login user) accessing the slave server 20. The login process is similar to the login process to the master server 10 described in the image check request process (see FIG. 21).

The controller 21 of the slave server 20 shows the examination list screen on the display 43 of the client terminal 40 (Step S11). Specifically, the controller 21 reads out the data of each examination from the examination table T11, etc. in the slave database 241, and generates data for displaying the examination list screen.

Here, the controller 21 refers to the check request task queue table T16 in the slave database 241 and extracts a record of the user ID associated with the login user in the “request target” column (Step S12).

If there is a record of the user ID associated with the login user in the “request target” column, the controller 21 displays a check request notification screen on the display 43 of the client terminal 40 (Step S13). Specifically, the controller 21 acquires the record of the user ID associated with the login user in the “request target” column from the check request task queue table T16 in the slave database 241, and specifies the “examination LID.” The controller 21 acquires the examination information (examination ID, examination date and time, etc.) associated with the specified “examination LID” and the patient information (patient name, age, sex, etc.), and generates data for displaying the check request notification screen.

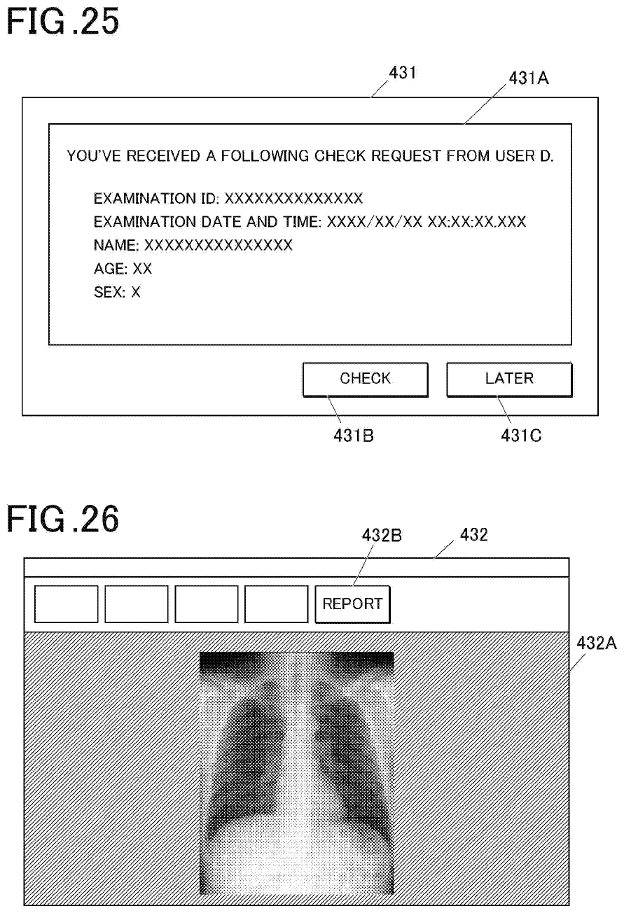

FIG. 25 shows an example of the check request notification screen 431 displayed on the display 43 of the client terminal 40. The check request notification screen 431 includes a check request notification area 431A, a “check” button 431B, and a “later” button 431C. In the check request notification area 431A, a notification of a request for checking an examination is shown to the login user.

When the user, on the client terminal 40, presses the check button 431B via the operation interface 42 (Step S14), the controller 21 of the slave server 20 adds a record to the check request response queue table T22 in the temporary report storage database 251 (Step S15). Specifically, the controller 21 assigns a new “check request response queue LID” in the check request response queue table T22, and the “examination LID,” the “request source,” the “request target,” and the “request date and time” are the same as those of the record acquired from the check request task queue table T16 (record of the user ID associated with the login user in the “request target” column).

The controller 11 of the master server 10 regularly polls the check request response queue table T22 in the temporary report storage database 251 of the slave server 20 via the communication unit 13 for constant monitoring (Step S16).

If there is a record added to the check request response queue table T22, the controller 11 of the master server 10 acquires the record added in the check request response queue table T22 added from the slave server 20 via the communication unit 13 (Step S17).

Next, the controller 11 of the master server 10 determines whether the record that has the same “request source” and “request target” as the record added to the check request response queue table T22 is in the check request task queue table T16 in the master database 141 (Step S18).

If the record that has the same “request source” and “request target” as the record added to the check request response queue table T22 is in the check request task queue table T16 in the master database 141, the controller 11 of the master server 10 deletes the concerning record from the check request task queue table T16 (Step S19).

The data in the master database 141 of the master server 10 is replicated in the slave database 241 of the slave server 20, and thus the check request task queue table T16 in the slave database 241 is updated (Step S20).

After Step S15, the controller 21 of the slave server 20 displays the viewer screen of the medical image(s) concerning the check request on the display 43 of the client terminal 40 (Step S21). Specifically, the controller 21 acquires the “series ID” associated with the “examination LID” included in the record added to the check request queue table T22 in the temporary report storage database 251 from the series table T12 in the slave database 241, acquires the “image LID” and the “image file path” associated with the acquired “series LID” from the image table T13 in the slave database 241, and generates data for displaying the viewer screen concerning the medical image(s) in the check request based on the acquired “image file path.”

FIG. 26 shows an example of the viewer screen 432 displayed on the display 43 of the client terminal 40. The viewer screen 432 includes a medical image display area 432A and a “report” button 432B. The medical image(s) concerning the check request is shown on the medical image display area 432A.

When the user, on the client terminal 40, presses the report button 432B via the operation interface 42 (Step S22), the controller 21 of the slave server 20 displays the report screen on the display 43 of the client terminal 40 (Step S23).

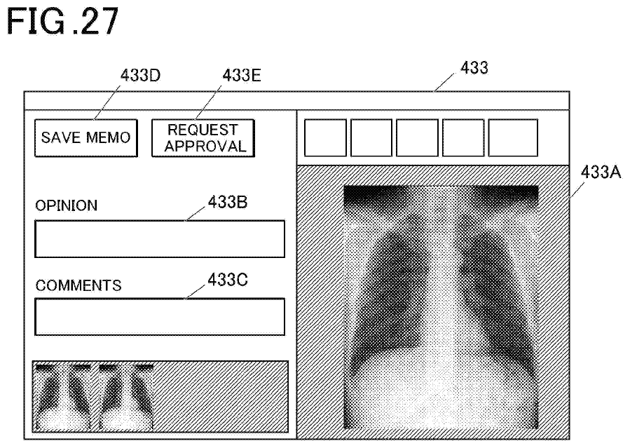

FIG. 27 shows an example of the report screen 433 displayed on the display 43 of the client terminal 40. The report screen 433 includes a medical image display area 433A, an opinion area 433B, a comment area 433C, a “save memo” button 433D, and a “request approval” button 433E.

The save memo button 433D is pressed when the temporary report is saved as a memo.

The request approval button 433E is pressed to request approval for saving the temporary report as a proper image interpretation report.

When the user, on the client terminal 40, inputs an opinion or comments via the operation interface 42, the input content is sent to the slave server 20.

The controller 21 of the slave server 20 generates a temporary report configuration file based on the input content (Step S24), and stores it in the temporary report storage 25. Specifically, the controller 21 generates a temporary report configuration file including a report text, a report configuration data, and an image(s) for report.

The controller 21 of the slave server 20 adds a record to the temporary report storage table T21 of the temporary report storage database 251 (Step S25). Specifically, the controller 21 assigns a new “temporary report LID” as a new record. The controller 21 enters the examination LID of the examination concerning the temporary in the “examination LID” column, the user ID of the login user in the “temporary report creator” column, the date and time of creation of the temporary report configuration file (present date and time acquired from the clock unit 26) in the “report last update date and time” column, the storage location of the report text file included in the temporary report configuration file in the “report text file path” column, and the storage location of the temporary report configuration file in the “report configuration file path” column. The controller 21 enters the viewable range (EVERYONE/ROLE/USER) designated by the login user in the “viewable range” column. The controller 21 then enters the role ID of the role designated by the login user (the user who is granted the permission for view) in the “role with view permission” column if the viewable range is “ROLE,” and enters the user ID of the user designated by the login user (the user who is granted the permission for view) in the “user with view permission” column. If the save memo button 433D is pressed on the report screen 433, the controller 21 leaves the “report status transition slave control definition ID” column blank, and if the request approval button 433E is pressed on the report screen 433, the controller 21 enters “1” in the “report status transition slave control definition ID.”

If the login user is the “image interpretation approval doctor (role ID: 2),” the approval button is provided instead of the approval request button 433E on the report screen 433. If the approval button is pressed on the report screen 433, the controller 21 enters “0” in the “report status transition slave control definition ID” column.

The temporary report creation process is now completed.

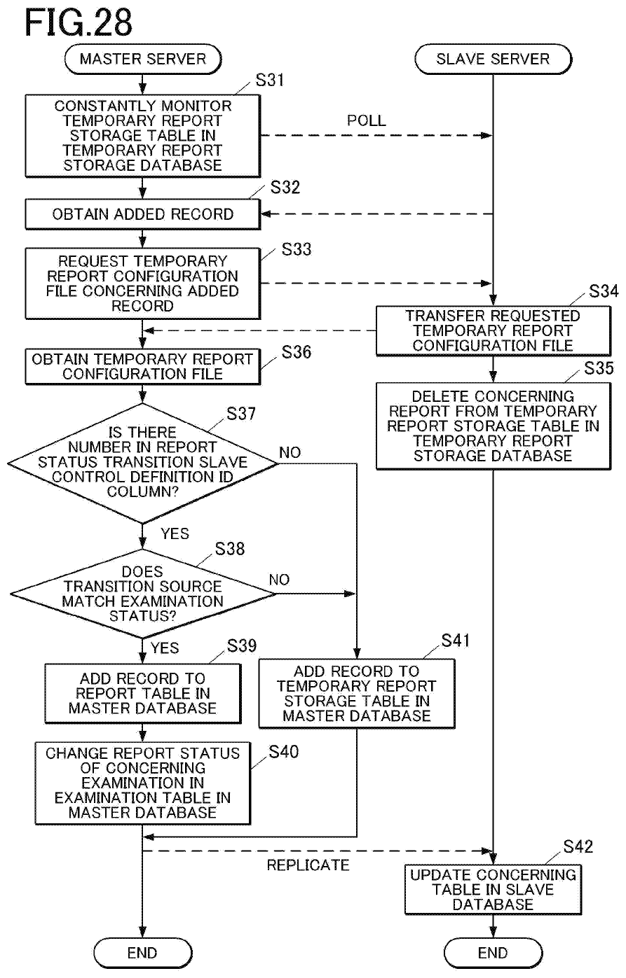

FIG. 28 is a ladder chart showing the temporary report acquisition request process. In the temporary report acquisition request process, the master server 10 checks the temporary report storage database 251 of the slave server 20 and takes in a new temporary report if present.

The controller 11 of the master server 10 regularly polls the temporary report storage table T21 in the temporary report storage database 251 of the slave server 20 via the communication unit 13 for constant monitoring (Step S31).

If there is a record added to the temporary report storage table T21, the controller 11 of the master server 10 acquires the record added to the temporary report storage table T21 from the slave server 20 via the communication unit 13 (Step S32).

Next, the controller 11 of the master server 10 requests the slave server 20 for the temporary report configuration file corresponding to the added record via the communication unit 20 (Step S33).

The controller 21 of the slave server 20 reads out the temporary report configuration file from the temporary report storage 25 based on the “report configuration file path” of the record added to the temporary report storage table T21 and transfers the requested temporary report configuration file to the master server 10 via the communication unit 23 (Step S34).

Next, the controller 21 of the slave server 20 deletes the record corresponding to the temporary report configuration file transferred to the master server 10 from the temporary report storage table T21 of the temporary report storage database 251 (Step S35).

The controller 11 of the master server 10 acquires the temporary report configuration file via the communication unit 13 (Step S36), and stores it in the storage 14 as a report configuration file.

The controller 11 of the master server 10 then determines whether a number is in the “report status transition slave control definition ID” column of the record acquired at Step S32 (Step S37).

If a number is in the “report status transition slave control definition ID” column (Step S37; YES), the controller 11 of the master server 10 acquires the value (report status) in the “transition source” associated with the “report status transition slave control definition ID” of the record acquired at Step S32 from the report status transition status slave control definition table T7 in the master database 141. The controller 11 determines whether the value in the “transition source” column matches the “report status” associated with the “examination LID” of the concerning examination in the examination table T11 in the master database 141 (Step S38).

If the value in the “transition source” column matches the “report status” of the concerning examination (Step S38: YES), the controller 11 of the master server 10 adds a record in the report table T14 in the master database 141 (Step S39). Specifically, the controller 11 assigns a new “report LID” in the report table T14 as a new record. The controller 11 enters the “examination LID” of the record acquired at Step S32 in the “examination LID” column, the “temporary report creator” of the record acquired at Step S32 in the “report creator” column and the “last report editor” column, and the date included in the “last report update date and time” of the record acquired at Step S32 in the “last report update date” column. The controller 11 enters the file path of the report configuration file (the temporary report configuration file acquired from the slave server 20) stored in the storage 14 in the “report configuration file path” column and the file path of the report text included in the report configuration file in the “report text file path” column. If the “report status transition slave control definition ID” of the record acquired at Step S32 is “0,” the controller 11 enters the “temporary report creator” of the record acquired at Step S32 in the “report approver” column.

The controller 11 of the master server 10 changes the “report status” of the concerning examination in the examination table T11 in the master database 141 (Step S40). Specifically, the controller 11 acquires the “transition target” associated with the “report status transition slave control definition ID” of the record acquired at Step S32 from the report status transition slave control definition table T7 in the master database 141, and enters the “report status” indicated by the acquired “transition target” in the “report status” column associated with the “examination LID” of the record acquired at Step S32 in the examination table T11 in the master database 141.

If no number is in the “report status transition slave control definition ID” column at Step S37 (Step S37; NO), or if the value in the “transition source” column does not match the “report status” of the concerning examination (Step S38; NO), the controller 11 of the master server 10 adds a record to the temporary report storage table T15 in the master database 141 (Step S41). Specifically, the controller 11 adds a record acquired at Step S32 to the temporary report storage table T15. The controller 11 enters the file path of the report configuration file (the temporary report configuration file acquired from the slave server 20) stored in the storage 14 in the storage 14, and enters the file path of the report text included in the report configuration file in the “report text file path” column.

The data in the master database 141 of the master server 10 is constantly replicated in the slave database 241 of the slave server 20, and thus the examination table T11, the report table T14, and the temporary report storage table T15 in the slave database 241 are also updated after Step S40 or S41 (Step S42).

The temporary report acquisition request process is now completed.

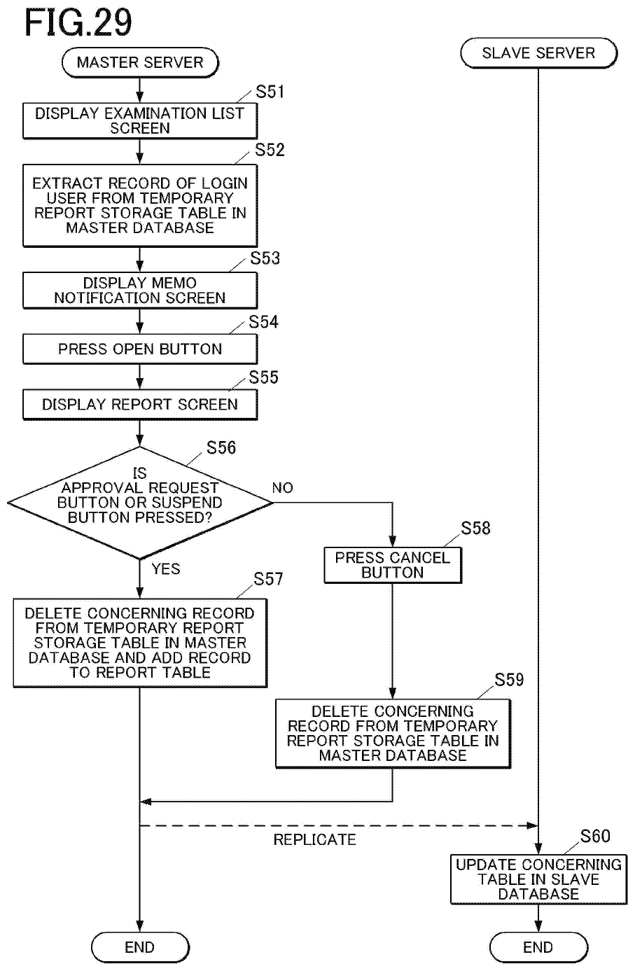

FIG. 29 is a ladder chart showing the temporary report check process. In the temporary report check process, the temporary report taken in from the slave server to the master server 10 20 is registered as a proper report.

As a premise of this process, the master server 10 is accessed from the client terminal 30 in the medical facility for the login process. The controller 11 of the master server 10 specifies the user (login user) accessing the master server 10. The login process is similar to that in the image check request process (see FIG. 21).

The controller 11 of the master server 10 displays the examination list screen on the display 33 of the client terminal 30 (Step S51).

The controller 11 extracts the record with the user ID associated with the login user in the “temporary report creator” from the temporary report storage table T15 in the master database 141 (Step S52).

Next, the controller 11 of the master server 10 displays the memo notification screen on the display 33 of the client terminal 30 (Step S53). Specifically, the controller 11 acquires the record with the user ID associated with the login user in the “temporary report creator” from the temporary report storage table T15 in the master database 141, and specifies the “examination LID.” The controller 11 acquires the examination information (examination ID, examination date and time, etc.) and the patient information (patient name, age, sex, etc.) associated with the specified “examination LID” and generates the data for displaying the memo notification screen.

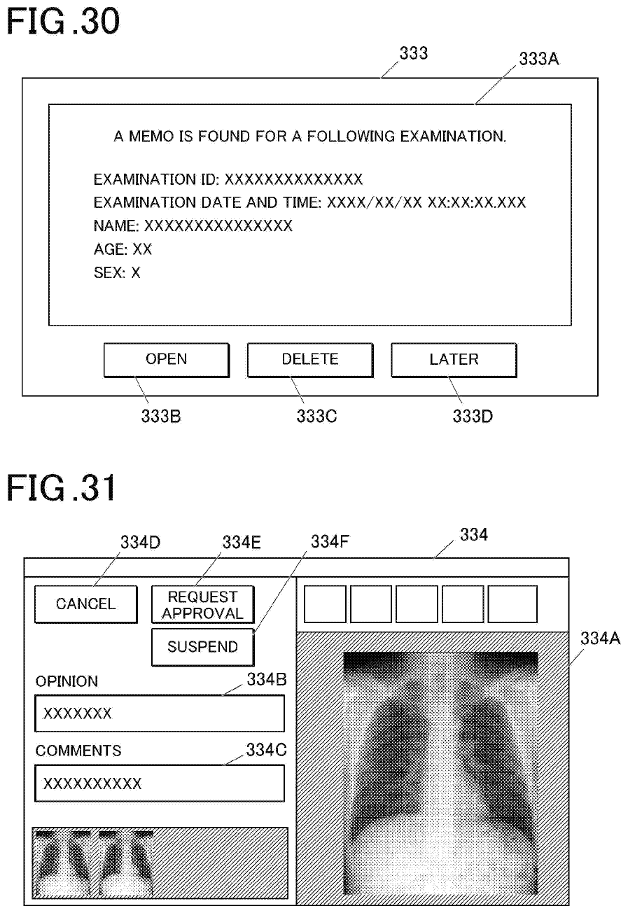

FIG. 30 shows an example of the memo notification screen displayed on the display 33 of the client terminal 30. On the memo notification screen 333, a memo notification area 333A, an “open” button 333B, a “delete” button 333C, and a “later” button 333D. In the memo notification area 333A, a notification of a memo created by the login user is shown.

When the user, on the client terminal 30, presses the open button 333B via the operation interface 32 (Step S54), the controller 11 of the master server 10 displays the report screen on the display 33 of the client terminal 30 (Step S55).

FIG. 31 shows an example of the report screen 334 displayed on the display 33 of the client terminal 30. The report screen 334 includes a medical image display area 334A, an opinion area 334B, a comment area 334C, a “cancel” button 334D, a “request approval” button 334E, and a “suspend” button 334F.

When the user, on the client terminal 30, presses the request approval button 334E or the suspend button 334F via the operation interface 32 (Step S56; YES), the controller 11 of the master server 10 deletes the concerning record (the record corresponding to the report checked at Step S55) from the temporary report storage table T15 in the master database 141 and adds a record to the report table T14 in the master database 141 (Step S57). Specifically, the controller 11 assigns a new “report LID” in the report table T14 as a new record. The controller 11 enters the “examination LID” of the record deleted from the temporary report storage table T15 in the “examination LID” column, the “temporary report creator” of the record deleted from the temporary report storage table T15 in the “report creator” column and the “last report editor” column, and the date included in the “last report update date and time” of the record deleted from the temporary report storage table T15 in the “last report update date” column. The controller 11 enters the “report configuration file path” of the record deleted from the temporary report storage table T15 in the “report configuration file path” column, and the “report text file path” of the record deleted from the temporary report storage table T15 in the “report text file path” column.

When the user, on the client terminal 30, presses the cancel button 334D via the operation interface 32 (Step S56; NO, Step S58), the controller 11 of the master server 10 deletes the concerning record (the record corresponding to the report checked at Step S55) from the temporary report storage table T15 in the master database 141 (Step S59).

The data in the master database 141 of the master server 10 is constantly replicated in the slave database 241 of the slave server 20, and thus the report table T14 and the temporary report storage table T15 in the slave database 241 are also updated (Step S60).

The temporary report check process is now completed.

As described hereinbefore, according to one or more embodiments of the present invention, it is possible to take in the report created outside the medical facility to the master server 10 inside the medical facility, while maintaining the consistency of data in the master server 10 inside the medical facility and the slave server 20 outside the medical facility.

This system can uniformly manage the report creation and approval flow in the two servers. In this system, a report and memo written from outside the medical facility with reference to the image(s) in the slave server 20 is saved as a temporary report and taken into the master server 10 in the medical facility, and can be used for a formal report. Therefore, the effort and time of report creation may be reduced.

The information for specifying the transition target of the report status that is set when the master server 10 takes in the report status is included in the processing request acquired by the master server 10 from the slave server 20. Thus, the transition target of the report status may be determined according to the request for processing in the master server 10.

The controller 21 of the slave server 20 creates the request for processing according to the user operation on the client terminal 40 outside the medical facility. Thus, the user may designate the transition target of the report status that is set when the master server 10 takes in the temporary report.

The above description is merely an example of the report management system according to one or more embodiments of the present invention, and does not limit the scope of the invention. The detailed configurations and operations of the components of the system can also be appropriately modified within the scope of the present invention.

For example, the user who created the report, the type of the medical image(s) to be interpreted, the report creation and approval flow may be changed according to the rules in the medical facility, the object, the type of the client terminal 40 used in the image interpretation, etc. when the temporary report is taken in to the master server 10 from the slave server 20.

The user who created the report on the client terminal 40 via the slave server 20 may directly designate the transition target.

The data format of the temporary report created in the slave server 20 may be modified according to the usage of the report data created outside the medical facility in the master server 10.

For example, if the report data created outside the medical facility is to be directly taken in, the temporary report is created in the report data format similar to that in the master server 10 in the slave server 20.

If a situation and record of diagnosis outside the medical facility is to be attached when a report is newly created inside the medical facility, a temporary report is created in the image or PDF format in the slave server 20.

If text data made outside the medical facility is to be copied and used when a report is newly created inside the medical facility, a temporary report is created in the text format in the slave server 20.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A report management system comprising:
a master server that manages a report created in a medical facility according to multiple states defined in a report creation and approval flow; and
a slave server that manages a duplicate of data managed in the master server and accessible from outside the medical facility,
wherein the slave server comprises:
a memory that stores a temporary report created based on an access from outside the medical facility; and
a first hardware processor that generates a request for processing that is executed when the master server takes in the temporary report, and
wherein the master server comprises:
a second hardware processor that:
acquires the temporary report and the request for processing of the temporary report from the slave server,
registers the acquired temporary report as the report to be managed based on the acquired request for processing, and
changes a state of the report to be managed to a state that is selected from among the multiple states based on the request for processing.

2. The report management system according to claim 1,
wherein the request for processing includes information specifying the state of the report, the state being selected when the master server takes in the temporary report.

3. The report management system according to claim 1,
wherein the multiple states include: undone; in progress; approved; approval pending; rejected; and approving.

4. The report management system according to claim 1,
wherein the first hardware processor generates the request for processing based on a user operation on a client terminal outside the medical facility.
